(19) United States
(12) Patent Application Publication
(10) Pub. No.: US 2025/0066274 A1
(43) Pub. Date: Feb. 27, 2025

(54) INTEGRATED PROCESSES UTILIZING WATER ELECTROLYSIS AND OXIDATIVE DEHYDROGENATION OF ETHANE

(71) Applicant: Chevron Phillips Chemical Company LP, The Woodlands, TX (US)

(72) Inventors: James L. Hillier, KINGWOOD, TX (US); Jonathan A. Powell, KINGWOOD, TX (US); Michael S. Webster-Gardiner, KINGWOOD, TX (US)

(21) Appl. No.: 18/808,186

(22) Filed: Aug. 19, 2024

Related U.S. Application Data
(60) Provisional application No. 63/520,948, filed on Aug. 22, 2023.

(51) Int. Cl. C07C 11/04; C01B 17/16; C01B 17/50; C07C 5/48; C07C 29/151; C07C 51/21; C07C 319/18; C08F 210/02; C10G 50/00; C25B 1/04; C25B 15/08

(52) U.S. Cl. CPC C07C 11/04; C01B 17/162; C01B 17/508; C07C 5/48; C07C 29/1516; C07C 51/21; C07C 319/18; C08F 210/02; C10G 50/00; C25B 1/04; C25B 15/081

(57) ABSTRACT

Processes for converting ethane into ethylene include the steps of subjecting a water feed stream to electrolysis to form O₂ and H₂, subjecting a mixture of ethane and O₂ to oxidative dehydrogenation to form a reaction product containing ethylene, acetic acid, water, and CO/CO₂, separating the reaction product into an ethylene product stream, an acetic acid product stream, a water product stream, and a gas stream containing CO/CO₂, and introducing the water product stream into the water feed stream for electrolysis. The ethylene product stream can be contacted with a suitable polymerization or oligomerization catalyst composition to produce ethylene polymers or ethylene oligomers.

### REFERENCE TO RELATED APPLICATION

This application claims the benefit of U.S. Provisional Patent Application No. 63/520,948, filed on Aug. 22, 2023, the disclosure of which is incorporated herein by reference in its entirety.

### FIELD OF THE INVENTION

The present disclosure generally relates to integrated electrolysis and oxidative dehydrogenation processes, and more particularly, relates to performing such processes for the conversion of ethane into ethylene.

### BACKGROUND OF THE INVENTION

Electrolysis of water produces O₂ and H₂ product streams. However, efficient use of these product streams in other chemical manufacturing processes can be challenging. It would be beneficial to use both the O₂ and H₂ product streams with minimal waste and to produce valuable chemical feedstocks. Accordingly, it is to these ends that the present invention is generally directed.

### SUMMARY OF THE INVENTION

This summary is provided to introduce a selection of concepts in a simplified form that are further described below in the detailed description. This summary is not intended to identify required or essential features of the claimed subject matter. Nor is this summary intended to be used to limit the scope of the claimed subject matter.

Aspects of this invention are directed to processes for converting ethane into ethylene. One such process can comprise (a) subjecting a water feed stream to electrolysis to form O₂ and H₂, (b) subjecting a mixture of ethane and all or any portion of the O₂ from step (a) to oxidative dehydrogenation to form a reaction product containing ethylene, acetic acid, water, and CO/CO₂, (c) separating the reaction product into an ethylene product stream, an acetic acid product stream, a water product stream, and a gas stream containing CO/CO₂, and (d) introducing all or any portion of the water product stream of step (c) into the water feed stream of step (a).

In accordance with another aspect of this invention, a process for converting methane is provided, and this aspect, the process can comprise (A) subjecting a water feed stream to electrolysis to form O₂ and H₂, (B) subjecting a mixture of methane and all or any portion of the O₂ from step (A) and CO₂ to autothermal reforming to form a reaction product containing H₂, CO, and water, (C) separating the reaction product into a H₂ product stream, a water product stream, and a gas stream containing CO/CO₂, and (D) introducing all or any portion of the water product stream of step (C) into the water feed stream of step (A).

In accordance with yet another aspect of this invention, a process is disclosed in which an electrolysis step and a gasification step are linked together. In this aspect, the process can comprise a) subjecting a water feed stream to electrolysis to form O₂ and H₂, and b) gasifying a mixture of a plastic and all or any portion of the O₂ from step a) to form a Syngas stream. Optionally, this process can further comprise a step of c) reacting a gas stream containing CO/CO₂ (e.g., from step (c) above) with all or any portion of the H₂ from step a) to form methanol. Alternatively, this process can further comprise the steps of c) separating CO and H₂ from the Syngas stream, and d) contacting the CO, H₂, and a multicomponent catalyst to form a reaction mixture containing ethanol.

Both the foregoing summary and the following detailed description provide examples and are explanatory only. Accordingly, the foregoing summary and the following detailed description should not be considered to be restrictive. Further, features or variations may be provided in addition to those set forth herein. For example, certain aspects may be directed to various feature combinations and sub-combinations described in the detailed description.

### BRIEF DESCRIPTION OF THE FIGURES

The following figures form part of the present specification and are included to further demonstrate certain aspects of the present invention. The invention may be better understood by reference to one or more of these figures in combination with the detailed description of specific embodiments presented herein.

FIGS. 1-6 are schematic flow diagrams of integrated processes for converting ethane into ethylene using electrolysis and oxidative dehydrogenation consistent with aspects of the present disclosure.

FIG. 7 illustrates different uses for oxygen that can be incorporated into the processes of FIGS. 1-6.

FIG. 8 illustrates a schematic flow diagram of autothermal reforming of methane that can be incorporated into the processes of FIGS. 1-7.

While the inventions disclosed herein are susceptible to various modifications and alternative forms, only a few specific embodiments have been shown by way of example in the drawings and are described in detail below. The figures and detailed descriptions of these specific embodiments are not intended to limit the breadth or scope of the inventive concepts or the appended claims in any manner. Rather, the figures and detailed written descriptions are provided to illustrate the inventive concepts to a person of ordinary skill in the art and to enable such person to make and use the inventive concepts.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2^{nd} Ed (1997), can be applied, as long as that definition does not conflict with any other disclosure or definition applied herein, or render indefinite or non-enabled any claim to which that definition is applied. To the extent that any definition or usage provided by any document incorporated herein by reference conflicts with the definition or usage provided herein, the definition or usage provided herein controls.

Herein, features of the subject matter are described such that, within particular aspects, a combination of different features can be envisioned. For each and every aspect and each and every feature disclosed herein, all combinations that do not detrimentally affect the processes or methods described herein are contemplated with or without explicit description of the particular combination. Additionally, unless explicitly recited otherwise, any aspect or feature disclosed herein can be combined to describe inventive processes or methods consistent with the present disclosure.

Generally, groups of elements are indicated using the numbering scheme indicated in the version of the periodic table of elements published in Chemical and Engineering News, 63 (5), 27, 1985. In some instances, a group of elements can be indicated using a common name assigned to the group; for example, alkali metals for Group 1 elements, alkaline earth metals for Group 2 elements, transition metals for Group 3-12 elements, and halogens or halides for Group 17 elements.

The term “hydrocarbon” whenever used in this specification and claims refers to a compound containing only carbon and hydrogen, whether saturated or unsaturated. Other identifiers can be utilized to indicate the presence of particular groups in the hydrocarbon (e.g., halogenated hydrocarbon indicates the presence of one or more halogen atoms replacing an equivalent number of hydrogen atoms in the hydrocarbon). Non-limiting examples of hydrocarbons include alkanes (linear, branched, and cyclic), alkenes (olefins), and aromatics, among other compounds.

For any particular compound or group disclosed herein, any name or structure (general or specific) presented is intended to encompass all conformational isomers, regioisomers, stereoisomers, and mixtures thereof that can arise from a particular set of substituents, unless otherwise specified. The name or structure (general or specific) also encompasses all enantiomers, diastereomers, and other optical isomers (if there are any) whether in enantiomeric or racemic forms, as well as mixtures of stereoisomers, as would be recognized by a skilled artisan, unless otherwise specified. For instance, a general reference to pentane includes n-pentane, 2-methyl-butane, and 2,2-dimethylpropane; and a general reference to a butyl group includes a n-butyl group, a sec-butyl group, an iso-butyl group, and a t-butyl group.

Unless otherwise specified, the term “substituted” when used to describe a group, for example, when referring to a substituted analog of a particular group, is intended to describe any non-hydrogen moiety that formally replaces a hydrogen in that group, and is intended to be non-limiting. Also, unless otherwise specified, a group or groups can also be referred to herein as “unsubstituted” or by equivalent terms such as “non-substituted,” which refers to the original group in which a non-hydrogen moiety does not replace a hydrogen within that group. Moreover, unless otherwise specified, “substituted” is intended to be non-limiting and include inorganic substituents or organic substituents as understood by one of ordinary skill in the art.

The terms “contacting” and “combining” are used herein to describe catalysts, compositions, processes, and methods in which the materials or components are contacted or combined together in any order, in any manner, and for any length of time, unless otherwise specified. For example, the materials or components can be blended, mixed, slurried, dissolved, reacted, treated, impregnated, compounded, or otherwise contacted or combined in some other manner or by any suitable method or technique.

In this disclosure, while processes and methods are described in terms of “comprising” various components or steps, the processes and methods also can “consist essentially of” or “consist of” the various components or steps, unless stated otherwise. The terms “a,” “an,” and “the” are intended to include plural alternatives, e.g., at least one, unless otherwise specified.

Several types of ranges are disclosed in the present invention. When a range of any type is disclosed or claimed, the intent is to disclose or claim individually each possible number that such a range could reasonably encompass, including end points of the range as well as any sub-ranges and combinations of sub-ranges encompassed therein. For example, an oligomerization process using ethylene as the monomer produces a mixture of products that contains various amounts of ethylene oligomers. By a disclosure that the amount of oligomers in the mixture of products can be at least 30 wt. % (and up to 100 wt. %), the intent is to recite that the weight percentage of oligomers can be any amount in the range and, for example, can include any range or combination of ranges from 30 wt. % to 100 wt. %, such as at least 50 wt. %, at least 60 wt. %, or at least 70 wt. %, and so forth. Likewise, all other ranges disclosed herein should be interpreted in a manner similar to this example.

In general, an amount, size, formulation, parameter, range, or other quantity or characteristic is “about” or “approximate” whether or not expressly stated to be such. Whether or not modified by the term “about” or “approximately,” the claims include equivalents to the quantities or characteristics.

Although any methods, devices, and materials similar or equivalent to those described herein can be used in the practice or testing of the invention, the typical methods, devices, and materials are herein described.

All publications and patents mentioned herein are incorporated herein by reference in their entirety for the purpose of describing and disclosing, for example, the constructs and methodologies that are described in the publications and patents, which might be used in connection with the presently described invention.

### DETAILED DESCRIPTION OF THE INVENTION

The present invention is generally directed, in one aspect, to the integration of (i) an electrolysis process and (ii) an oxidative dehydrogenation of ethane to produce ethylene. Renewable electricity generated by wind and solar energy have the potential to produce commercially competitive hydrogen through the electrolysis of water. Splitting of a water molecule will produce hydrogen and oxygen in weight ratio of roughly 1:8. This excess oxygen can be used efficiently for a variety of manufacturing processes. For instance, oxygen can be used as a reactant in an oxidative hydrogenation (ODH) process over mixed metal oxide catalysts, which is an exothermic process. One by-product of ODH is water, which is an input to the electrolysis process. Excess oxygen also can be used for gasification of plastics to produce Syngas and its derivatives, partial oxidation reactions of mercaptans to sulfides, and to enrich fired process applications to increase efficiency and promote carbon capture. Further, by-product carbon dioxide from the ODH process also can be used for the synthesis of methanol using the reverse gas shift reaction and other Syngas derivatives.

A key objective of the present invention is to couple two processes in order to take advantage of the by-products of each to generate feedstocks for the other. For instance, the oxygen from electrolysis can be used in various chemical processes, such as the ODH of ethane. The OHD of ethane, in turn, provides water for the electrolysis process. Additionally, heat from the ODH process can be used in the electrolysis process (reducing the amount of electricity needed for solid oxide electrolyzer cells), as well as in other chemical processes.

Another key objective is to use the ethylene from the ODH of ethane to produce ethylene polymers, such as polyethylene, and ethylene oligomers, such as 1-hexene and 1-octene.

Another key objective is to use ODH by-products, such as CO/CO₂, with the hydrogen product stream from electrolysis to generate methanol.

Another key objective is to use the hydrogen product stream from electrolysis as a reactant that can be combined with sulfur to form H₂S. In the sulfur chemical area, a related objective is to use the oxygen product stream from electrolysis as a reactant that can be combined with sulfur to form SO₂ (or combined with methyl mercaptan to form dimethyl disulfide).

Another key objective is to utilize water electrolysis and cryogenic air separation to generate hydrogen, oxygen, and nitrogen, and to utilize the hydrogen and nitrogen to produce ammonia. Further, CO₂ generated in the ODH process can be reacted with ammonia to produce urea, which is an important fertilizer feedstock.

Another key objective is to use the water by-product from ODH to reduce or eliminate the water demand for the electrolysis process, particularly if the combined electrolysis and ODH processes are in locations where sufficient sources of useable water are not readily available.

Processes for Converting Ethane into Ethylene

Disclosed herein are processes for producing ethylene from ethane, and these processes can comprise (or consist essentially of, or consist of) (a) subjecting a water feed stream to electrolysis to form O₂ and H₂, (b) subjecting a mixture of ethane and all or any portion of the O₂ from step (a) to oxidative dehydrogenation to form a reaction product containing ethylene, acetic acid, water, and CO/CO₂, (c) separating the reaction product into an ethylene product stream, an acetic acid product stream, a water product stream, and a gas stream containing CO/CO₂, and (d) introducing all or any portion of the water product stream of step (c) into the water feed stream of step (a).

Generally, the features of the processes for producing ethylene from ethane (e.g., the electrolysis step, the oxidative dehydrogenation step, the separating step, the water introduction step, among others) are independently described herein and these features can be combined in any combination to further describe the disclosed processes to produce ethylene from ethane. Moreover, additional process steps can be performed before, during, and/or after any of the steps in any of the processes disclosed herein and can be utilized without limitation and in any combination to further describe these processes, unless stated otherwise. Further, any ethylene product streams produced in accordance with the disclosed processes are within the scope of this disclosure and are encompassed herein.

Referring first to step (a), the water feed stream is subjected to electrolysis to form O₂ and H₂. Any suitable source of electricity can be utilized to power the electrolysis process, but in some aspects, a green source of electricity, such as wind energy, solar energy, and the like, can be used to conduct the electrolysis of the water feed stream to form O₂ and H₂. Advantageously, heat generated in the oxidative dehydrogenation of step (b) can be used in the electrolysis step, the heat reducing the energy consumption needed for the electrolysis process. The heat generated also can be utilized in any downstream separations process (such as in step (c)) and for other process steps discussed further hereinbelow, as needed.

In step (b), a mixture of ethane and (all or any portion of) the O₂ from step (a) is subjected to oxidative dehydrogenation (ODH) to form a reaction product containing ethylene (greater than 80%), acetic acid (approximately 12%), water, and CO/CO₂ (less than 5%). In general, ODH uses oxygen and mixed metal oxide catalysts to produce olefins from respective alkanes, such as ethylene from ethane. Suitable catalysts and typical reaction conditions for step (b) are disclosed in “Process for producing ethylene via oxidative dehydrogenation (ODH) of ethane,” PCT international publication number WO 2010/115108, and “Oxidative dehydrogenation of ethane,” Linde Engineering, 2020 AIChE Spring National Meeting, paper number 124b, Mar. 30-Apr. 1, 2020. See also U.S. Patent Publication No. 2009/0292153, and U.S. Pat. Nos. 7,829,753 and 6,518,476.

In step (c), the reaction product is separated into an ethylene product stream, an acetic acid product stream, a water product stream, and a gas stream containing CO/CO₂. Any suitable technique can be used for this separating step, non-limiting examples of which include extraction, filtration, evaporation, distillation, and the like, as well as any combination thereof. Thus, combinations of two or more suitable separations techniques can be used in step (c).

In step (d), all or any portion of the water product stream of step (c) is introduced into the water feed stream of step (a). In some aspects, the water product stream supplies all the water required for the water feed stream of step (a), while in another aspects, a water make-up stream is combined with water product stream (e.g., in any suitable relative amount) to form the water feed stream of step (a).

The ethylene produced from ethane in the disclosed processes can be used to produce ethylene polymers and/or ethylene oligomers. For instance, the processes can further comprise a step of contacting a polymerization catalyst composition with all or any portion of the ethylene product stream—from step (c)—and an optional olefin comonomer in a polymerization reactor system under polymerization conditions to produce an ethylene polymer. Additionally or alternatively, the processes can further comprise a step of contacting an oligomerization catalyst composition with all or any portion of the ethylene product stream—from step (c)—in an oligomerization reactor system under oligomerization conditions to produce ethylene oligomers. Beneficially, both of the options are exothermic and therefore generate heat that can be used in the electrolysis step (as well other process steps discussed further hereinbelow).

Similar to the ethylene product stream in step (c), the H₂ produced from the electrolysis step (a) can be utilized in other chemical transformations. For example, the processes disclosed herein can further comprise a step of reacting sulfur with all or any portion of the H₂ from step (a) to form H₂S.

Likewise, the O₂ produced from the electrolysis step (a) can be utilized in chemical transformations in addition to the oxidative dehydrogenation of step (b). In an aspect, the processes disclosed herein can further comprise a step of reacting sulfur with a portion of the O₂ from step (a) to form SO₂ and/or the processes disclosed herein can further comprise a step of reacting methyl mercaptan with a portion of the O₂ from step (a) to form dimethyl disulfide.

Materials produced from different steps also can be combined to form valuable products. As a non-limiting example, the processes disclosed herein can further comprise a step of reacting all or any portion of the gas stream containing CO/CO₂ from step (c) with all or any portion of the H₂ from step (a) to form methanol. An illustrative technique for methanol synthesis is disclosed in U.S. Patent Publication No. 2023/0101490.

Referring again to step (c), in this step, the reaction product is separated into an ethylene product stream, an acetic acid product stream, a water product stream, and a gas stream containing CO/CO₂. In a variation of step (c), the reaction product can be separated into the ethylene product stream, a mixture of acetic acid and water, and the gas stream containing CO/CO₂, and the mixture (of acetic acid and water) can be separated into the acetic acid product stream, the water product stream, and steam. Optionally, all or any portion of the steam can be utilized in the oxidative dehydrogenation of step (b).

An air separation unit can be integrated with the disclosed processes for converting ethane into ethylene, such that the disclosed processes can further comprise a step of processing air through an air separation unit to form N₂ and O₂. Thus, there are two sources of O₂. All or any portion of the O₂ can be used in the oxidative dehydrogenation of step (b). Additionally or alternatively, all or any portion of the O₂ from the air separation unit can be utilized to produce SO₂ and/or dimethyl disulfide, as discussed above.

When an air separation unit is present, ammonia can be synthesized. In such circumstances, the processes disclosed herein further comprise a step of contacting all or any portion of H₂ from the electrolysis of step (a), all or any portion of the N₂ from the air separation unit, and an ammonia synthesis catalyst to produce ammonia.

In a further aspect, urea can be produced from ammonia. In this aspect, the processes disclosed herein can further comprise a step of contacting all or any portion of CO₂ from the gas stream containing CO/CO₂ of step (c) and all or any portion of the ammonia (which was produced from H₂ and N₂) to produce the urea. The urea, thus formed, can be used as a fertilizer, thus promoting the production of biomass products that can be used to form ethanol, for example, via fermentation.

Additionally or alternatively, ethanol can be synthesized. For ethanol production, the processes disclosed herein can further comprise a step of contacting all or any portion of CO₂ from the gas stream containing CO/CO₂ of step (c), all or any portion of the H₂ from the electrolysis of step (a), all or any portion of the acetic acid product stream of step (c), and a catalyst to from the ethanol product.

Referring now to aspects in which ethanol is produced, the ethanol can be utilized—if desired—to produce additional ethylene for use in ethylene polymerization and/or ethylene oligomerization processes. This can be achieved by contacting all or any portion of the ethanol with a catalyst to produce a reaction mixture containing ethylene and water, which can be performed in a vapor phase dehydration reactor. Suitable catalysts and reaction conditions are disclosed in U.S. Pat. No. 8,440,873. Subsequently, the reaction mixture can be separated into an ethylene stream and a water stream. This separating step provides a higher purity ethylene feed for use in the subsequent polymerization/oligomerization process steps. The separating step can include any suitable technique or any technique disclosed herein, such as extraction, filtration, evaporation, distillation, and the like, as well as combination of two or more techniques. All or any portion of the ethylene stream can be combined with the ethylene product stream of step (c), e.g., for subsequent polymerization and/or oligomerization, and all or any portion of the water stream can be combined with the water feed stream of step (a) and subjected to electrolysis.

In step (c), the reaction product can be separated into the ethylene product stream, the acetic acid product stream, the water product stream, and the gas stream containing CO/CO₂. If desired, the processes disclosed herein can further comprise a step of subjecting all or any portion of the CO/CO₂ stream to carbon capture and storage (CCS) to isolate CO₂.

Referring again to step (a), electrolysis of the water feed stream produces O₂ and H₂. In addition to oxidative dehydrogenation in step (b), the O₂ can be used in various other processes. For instance, a mixture of any portion of the O₂ from step (a) and a plastic (biomass, mixed solid waste, or a combination thereof) can be gasified to form a Syngas stream. Additionally or alternatively, a mixture of any portion of the O₂ from step (a) and a plastic (biomass, mixed solid waste, or a combination thereof) can be gasified and subjected to a Fischer-Tropsch process to produce alkanes (e.g., for fuel applications). Additionally or alternatively, a mixture of any portion of the O₂ from step (a) and a plastic (biomass, mixed solid waste, or a combination thereof) can be gasified to form a Syngas stream, and then reacting all or any portion of the gas stream containing CO/CO₂ from step (c) with all or a portion of the H₂ from step (a) to form methanol. Additionally or alternatively, the processes disclosed herein can further comprise (i) gasifying a mixture of any portion of the O₂ from step (a) and a plastic to form a Syngas stream, (ii) separating CO and H₂ from the Syngas stream, and (iii) contacting the CO, H₂, and a multicomponent catalyst to form a reaction mixture containing ethanol. An illustrative and non-limiting example of the multicomponent catalyst that can be used in step (iii) can include a potassium-modified ZnO—ZrO₂, modified zeolite mordenite, and Pt—Sn/SiC. Other suitable catalysts and typical reaction conditions for step (iii) are disclosed in Kang et al., “Single-pass transformation of syngas into ethanol with high selectivity by triple tandem catalysts,” Nature Communications, 2020, 11:827.

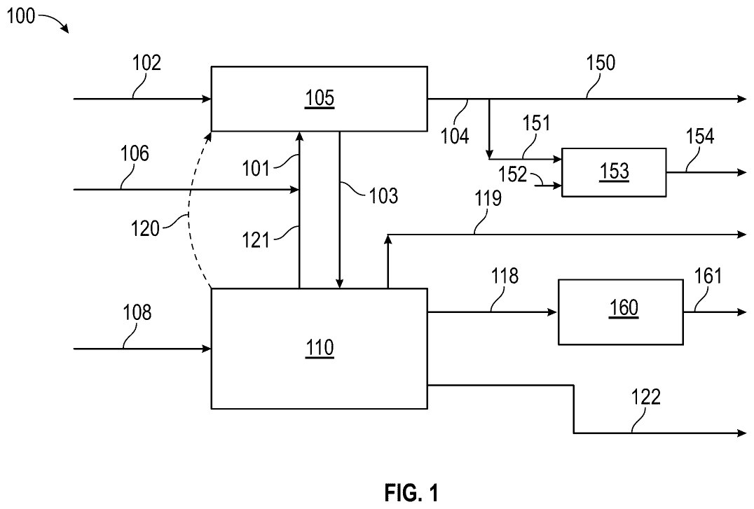

Referring now to FIG. 1, which illustrates a schematic flow diagram of process 100 for converting ethane into ethylene consistent with an aspect of the present disclosure. Water feed stream 101 is introduced into electrolysis unit 105 and electricity 102 is applied to form hydrogen stream 104 and oxygen stream 103. The oxygen stream 103 is fed into oxidative dehydrogenation (ODH) unit 110 along with ethane feed stream 108. Product streams discharged from dehydrogenation unit 110 include water stream 121, acetic acid stream 122, CO/CO₂ stream 119, and ethylene stream 118. The ethylene stream 118 is polymerized in polymerization unit 160 to form ethylene polymer 161, such as polyethylene.

The electrolysis unit 105 in FIG. 1 supplies oxygen stream 103 to oxidative dehydrogenation unit 110, and in turn, oxidative dehydrogenation unit 110 supplies water to electrolysis unit 105. Water stream 121 is combined with make-up water 106 as needed to form water feed stream 101 to electrolysis unit 105. Further, the reaction of ethane and oxygen is exothermic, so the oxidative dehydrogenation unit 110 transfers heat 120 to heat water for improved efficiency of electrolysis unit 105. Additionally or alternatively, heat 120 can be used in other operations in the process 100 that may require thermal energy.

The hydrogen stream 104 that is discharged from electrolysis unit 105 can be used for a variety of purposes. A portion can be hydrogen product 150, while another can be hydrogen feedstock 151 that is combined with sulfur stream 152 in H₂S synthesis unit 153 to produce H₂S product stream 154. While not shown in FIG. 1, but similarly, a portion of oxygen stream 103 can be combined with methyl mercaptan to form dimethyl disulfide.

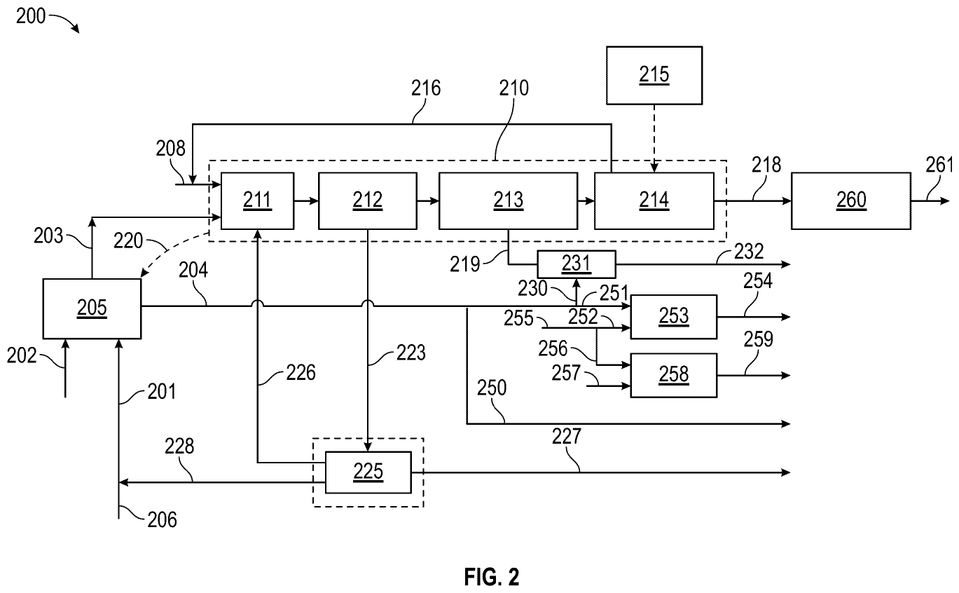

Referring now to FIG. 2, which illustrates an alternative process 200 for converting ethane into ethylene consistent with another aspect of the present disclosure. The reference numerals in FIG. 2 are generally the same as described for the similarly numbered components in FIG. 1, with the following exceptions. In FIG. 2, the various steps and/or unit operations that may be present in oxidative dehydrogenation unit 210 are shown: oxidative dehydrogenation reactor 211, hot section with water quench 212, gas removal 213, ethylene recovery 214, OHD refrigeration 215, and ethane recycle 216. CO/CO₂ stream 219 is discharged from gas removal 213 section of ODH unit 210 and combined with partial hydrogen stream 230 (from hydrogen stream 204) in methanol synthesis unit 231 to form methanol product stream 232. Water-acetic acid mixture 223 is discharged from hot section with water quench 212 and fed to acetic acid recovery unit 225, and discharged therefrom are acetic acid product stream 227, steam stream 226 (which can be recycled to ODH reactor 211), and water stream 228 (which can be combined with make-up water 206 to form water feed stream 201 to electrolysis unit 205).

A hydrogen feedstock 251 portion of hydrogen stream 204 that is discharged from electrolysis unit 205 is combined with sulfur stream 252 (which is a portion of sulfur feed 255), in H₂S synthesis unit 253 to produce H₂S product stream 254 in FIG. 2. The other portion of sulfur feed 255 is sulfur stream 256, which is combined with oxygen feed stream 257 in SO₂ synthesis unit 258 to form SO₂ product stream 259. All or any portion of oxygen feed stream 257 can come from oxygen stream 203 from electrolysis unit 205.

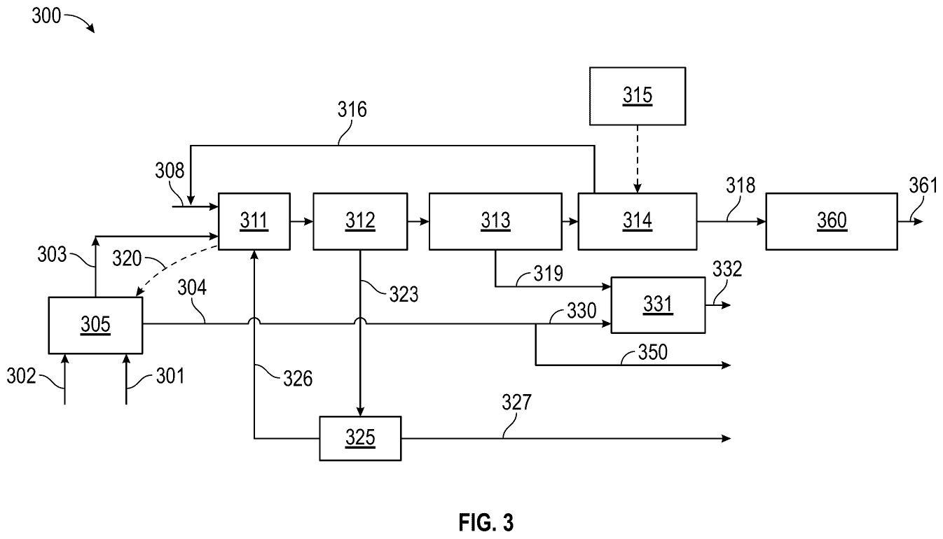

FIG. 3 illustrates an alternative process 300 for converting ethane into ethylene consistent with another aspect of the present disclosure. The reference numerals in FIG. 3 are generally the same as described for the similarly numbered components in FIGS. 1-2, with the following exceptions. In FIG. 3, the sulfur-based processes (H₂S synthesis unit, SO₂ synthesis unit) are omitted.

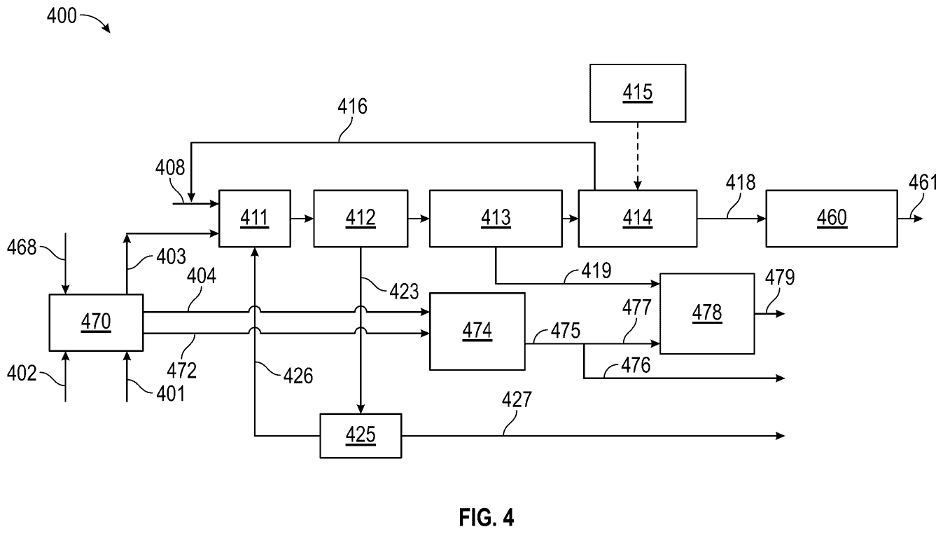

Referring to FIG. 4, which illustrates an alternative process 400 for converting ethane into ethylene consistent with another aspect of the present disclosure. The reference numerals in FIG. 4 are generally the same as described for the similarly numbered components in FIG. 3, with the following exceptions. In FIG. 4, electrolysis and air separation are collectively in separation unit 470. Water 401 is subjected to electrolysis in separation unit 470 to form hydrogen stream 404 and oxygen stream 403, and air 468 is processed through separation unit 470 to from nitrogen stream 472 and oxygen stream 403. The resulting nitrogen stream 472 and hydrogen stream 404 are catalytically converted in ammonia synthesis unit 474 to form ammonia effluent 475. A portion of ammonia effluent 475 is ammonia product 476 and another portion is ammonia feed 477, which is combined with CO₂ from CO/CO₂ stream 419 in urea synthesis unit 478 and converted into urea product stream 479.

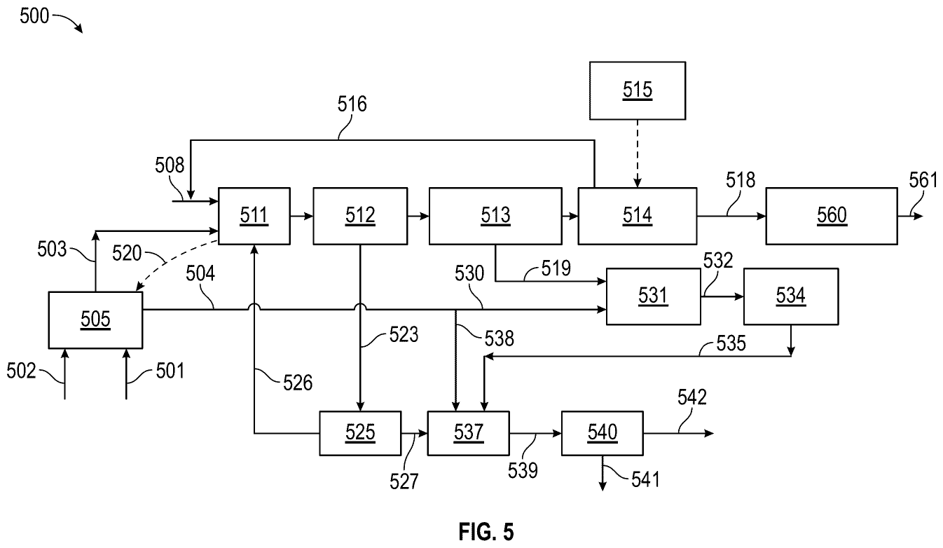

Referring to FIG. 5, which illustrates an alternative process 500 for converting ethane into ethylene consistent with another aspect of the present disclosure. The reference numerals in FIG. 5 are generally the same as described for the similarly numbered components in FIG. 3, with the following exceptions. In FIG. 5, methanol product stream 532 is catalytically converted in reactor unit 534 to acetic acid feedstock 535, and combined with acetic acid product stream 527 and partial hydrogen stream 538 (from hydrogen stream 504) in ethanol synthesis unit 537 to produce ethanol stream 539. Ethanol stream 539 is catalytically converted 540 to ethylene product 542 and discharged water 541. The ethylene product 542 can be combined with ethylene stream 518 and polymerized in polymerization unit 560 to form ethylene polymer 561, such as polyethylene. The discharged water 541 can be recycled into water feed stream 501 and introduced into electrolysis unit 505. In an aspect, the process 500 of FIG. 5 can be operated with a requirement of zero fresh water.

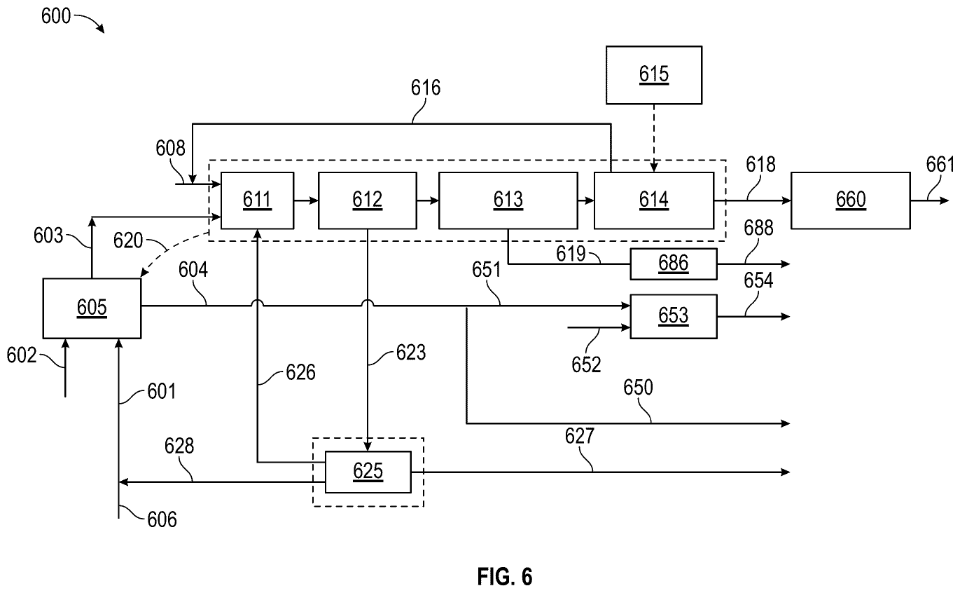

FIG. 6 illustrates an alternative process 600 for converting ethane into ethylene consistent with another aspect of the present disclosure. The reference numerals in FIG. 6 are generally the same as described for the similarly numbered components in FIG. 2, with the following exceptions. In FIG. 6, CO/CO₂ stream 619 is subjected to carbon capture and storage (CCS) 686 to isolate CO₂ stream 688.

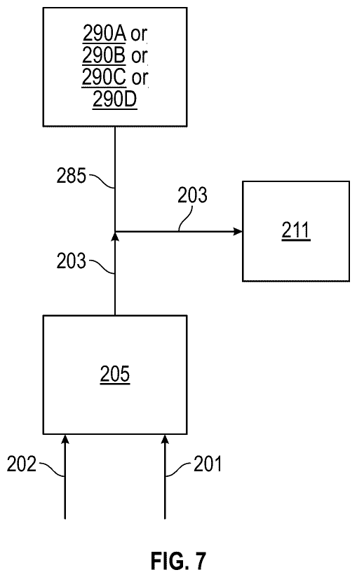

FIG. 7 shows a sub-unit variation of the processes illustrated in FIGS. 1-6 in which only a portion of the oxygen from electrolysis is supplied to the ODH unit. Another portion is utilized for other chemical processes or operations. FIG. 7 is described representatively in conjunction with FIG. 2, but it can be incorporated likewise into any of FIG. 1 and FIGS. 3-6. Water feed stream 201 in FIG. 7 is introduced into electrolysis unit 205 and electricity 202 is applied to form a hydrogen stream (not shown) and oxygen stream 203. Oxygen stream 203 is fed into oxidative dehydrogenation reactor 211 (as part of an oxidative dehydrogenation unit). A portion of oxygen stream 203 is oxygen feedstock 285, which can be used in a variety of chemical processes and operations that utilize oxygen: plastic gasification unit 290A, or plastic gasification and Fischer-Tropsch unit 290B, or plastic gasification and methanol unit 290C, or plastic gasification and ethanol to ethylene unit 290D, or any combination of these operations.

### Methane Conversion Processes

Disclosed herein are processes for converting methane. A representative process can comprise (A) subjecting a water feed stream to electrolysis to form O₂ and H₂, (B) subjecting a mixture of methane and (all or any portion of) the O₂ from step (a) and CO₂ to autothermal reforming (abbreviated ATR, also to be understood here as including partial oxidation) to form a reaction product containing H₂, CO, and water, (C) separating the reaction product into a H₂ product stream, a water product stream (if present), and a gas stream containing CO/CO₂, and (D) introducing (all or any portion of) the water product stream of step (C) into the water feed stream of step (A).

Generally, the O₂ from the electrolysis can be used for more H₂ generation from methane and extra water (if any) can be recycled back to the electrolysis. Also, any excess heat can be used to preheat the electrolysis water. In an aspect, ATR can produce water which is then further reacted to make additional H₂. For instance, CO and H₂O can be reacted to form CO₂+H₂, and the extent of this reaction can determine the net amount of water produced.

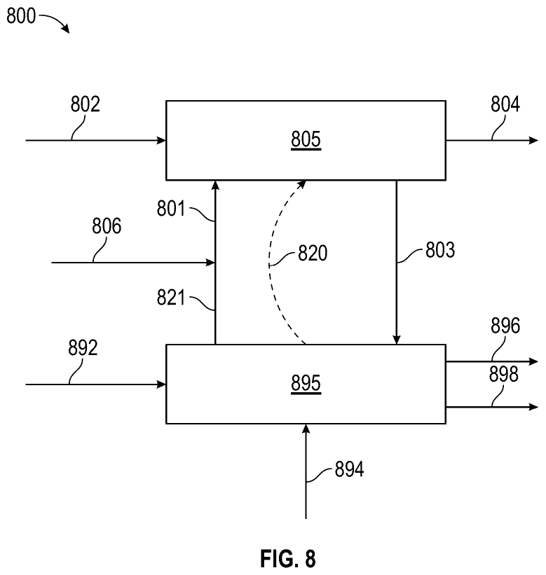

FIG. 8 shows a sub-unit variation of the processes illustrated in FIGS. 1-7 in which the oxidative dehydrogenation (OHD) unit is replaced with an autothermal reforming (ATR) unit for methane. FIG. 8 can be incorporated into any of FIGS. 1-7. Similar to representative FIG. 1, the process 800 of FIG. 8 includes water feed stream 801 that is introduced into electrolysis unit 805 and electricity 802 is applied to form hydrogen stream 804 and oxygen stream 803. Oxygen stream 803 is fed into ATR unit 895 along with methane feed stream 892 and carbon dioxide feed stream 894. Product streams discharged from ATR unit 895 include water stream 821, hydrogen product stream 896, and carbon monoxide stream 898.

The electrolysis unit 805 in FIG. 8 supplies oxygen stream 803 to ATR unit 895, and in turn, ATR unit 895 supplies water to electrolysis unit 805. Water stream 821 is combined with make-up water 806 as needed to form water feed stream 801 to electrolysis unit 805. Further, in an aspect, the ATR unit 895 can transfer heat 820 to heat water for improved efficiency of electrolysis unit 805.

### Ethylene Polymerization and Ethylene Oligomerization

As described herein, the ethylene produced from ethane in the disclosed processes can be used to produce ethylene polymers and/or ethylene oligomers. Thus, the processes can further comprise a step of contacting a polymerization catalyst composition with all or any portion of the ethylene product stream—from step (c)—and an optional olefin comonomer in a polymerization reactor system under polymerization conditions to produce an ethylene polymer. Additionally or alternatively, the processes can further comprise a step of contacting an oligomerization catalyst composition with all or any portion of the ethylene product stream—from step (c)—in an oligomerization reactor system under oligomerization conditions to produce ethylene oligomers.

Referring first to polymerization, a polymerization catalyst composition is contacted with the ethylene and an optional olefin comonomer in a polymerization reactor system under polymerization conditions to produce the ethylene polymer. Polymerization catalyst compositions, comonomer options, polymerization reactor systems and suitable reactor types, polymerization conditions, and resulting ethylene polymers are well known to those of skill in the art.

Briefly, polymerization catalyst compositions that are suitable for use include, but are not limited to, Ziegler-Natta based catalyst systems, chromium-based catalyst systems, metallocene-based catalyst systems, and the like, including combinations thereof. Hence, the polymerization catalyst composition can be a Ziegler-Natta based catalyst system, a chromium-based catalyst system, and/or a metallocene-based catalyst system; alternatively, a Ziegler-Natta based catalyst system; alternatively, a chromium-based catalyst system; or alternatively, a metallocene-based catalyst system. In one aspect, the polymerization catalyst composition can be a dual catalyst system comprising at least one metallocene compound, while in another aspect, the catalyst composition can be a dual catalyst system comprising two different metallocene compounds.

Examples of representative and non-limiting polymerization catalyst compositions include those disclosed in U.S. Pat. Nos. 3,887,494, 4,053,436, 4,981,831, 4,364,842, 4,444,965, 4,364,855, 4,504,638, 4,364,854, 4,444,964, 4,444,962, 3,976,632, 4,248,735, 4,297,460, 4,397,766, 2,825,721, 3,225,023, 3,226,205, 3,622,521, 3,625,864, 3,900,457, 4,301,034, 4,547,557, 4,339,559, 4,806,513, 5,037,911, 5,219,817, 5,221,654, 4,081,407, 4,296,001, 4,392,990, 4,405,501, 4,151,122, 4,247,421, 4,397,769, 4,460,756, 4,182,815, 4,735,931, 4,820,785, 4,988,657, 5,436,305, 5,610,247, 5,627,247, 3,242,099, 4,808,561, 5,275,992, 5,237,025, 5,244,990, 5,179,178, 4,855,271, 4,939,217, 5,210,352, 5,401,817, 5,631,335, 5,571,880, 5,191,132, 5,480,848, 5,399,636, 5,565,592, 5,347,026, 5,594,078, 5,498,581, 5,496,781, 5,563,284, 5,554,795, 5,420,320, 5,451,649, 5,541,272, 5,631,203, 5,654,454, 5,705,579, 5,668,230, 6,300,271, 6,831,141, 6,653,416, 6,613,712, 7,294,599, 6,355,594, 6,395,666, 6,833,338, 7,417,097, 6,548,442, 7,312,283, 7,026,494, 7,041,617, 7,199,073, 7,226,886, 7,517,939, 7,619,047, 7,919,639, and 8,080,681.

Such polymerization catalyst compositions, in addition to a transition metal, can contain an activator and an optional co-catalyst, and the catalyst system can be unsupported or supported on any suitable solid support (e.g., a porous solid oxide). Illustrative activators can include, but are not limited to, aluminoxane compounds (e.g., methylaluminoxane, MAO), organoboron or organoborate compounds, ionizing ionic compounds, activator-supports (e.g., a solid oxide treated with an electron-withdrawing anion), and the like, or combinations thereof. Commonly used polymerization co-catalysts can include, but are not limited to, organoaluminum and organozinc compounds, illustrative examples of which include trimethylaluminum (TMA), triethylaluminum (TEA), tri-n-propylaluminum (TNPA), tri-n-butylaluminum (TNBA), triisobutylaluminum (TIBA), tri-n-hexylaluminum, tri-n-octylaluminum, diisobutylaluminum hydride, diethylaluminum ethoxide, diethylaluminum chloride, dimethylzinc, diethylzinc (DEZ), dipropylzinc, dibutylzinc, dincopentylzinc, di(trimethylsilyl)zinc, di(tricthylsilyl)zinc, di(triisoproplysilyl)zinc, di(triphenylsilyl)zinc, di(allyldimethylsilyl)zinc, di(trimethylsilylmethyl)zinc, and the like, or combinations thereof.

Suitable olefin comonomers that can be polymerized (e.g., copolymerized, terpolymerized) with ethylene can include, but are not limited to, propylene, 1-butene, 2-butene, 3-methyl-1-butene, isobutylene, 1-pentene, 2-pentene, 3-methyl-1-pentene, 4-methyl-1-pentene, 1-hexene, 2-hexene, 3-ethyl-1-hexene, 1-heptene, 2-heptene, 3-heptene, 1-octene, 1-decene, styrene, and the like, or combinations thereof. According to one aspect, the olefin comonomer can comprise an α-olefin (e.g., a C₃-C₁₀ α-olefin), while in another aspect, the comonomer can comprise propylene, 1-butene, 1-pentene, 1-hexene, 1-octene, or any combination thereof; alternatively, the olefin comonomer can comprise 1-butene, 1-hexene, 1-octene, or a combination thereof; alternatively, the olefin comonomer can comprise 1-butene; alternatively, the olefin comonomer can comprise 1-hexene; or alternatively, the olefin comonomer can comprise 1-octene.

The polymerization reactor system can include any polymerization reactor capable of polymerizing ethylene and an olefin comonomer(s) (if used) to produce ethylene-based homopolymers, copolymers, terpolymers, and the like. The various types of polymerization reactors include those that can be referred to as a batch reactor, slurry reactor, gas-phase reactor, solution reactor, high pressure reactor, tubular reactor, autoclave reactor, and the like, or combinations thereof; or alternatively, the polymerization reactor system can comprise a slurry reactor (e.g., a loop slurry reactor), a gas-phase reactor (e.g., a fluidized bed reactor), a solution reactor, or a combination thereof. The polymerization reactor system can comprise a single reactor or multiple reactors (2 reactors, more than 2 reactors) of the same or different type. For instance, the polymerization reactor system can comprise a slurry reactor, a gas-phase reactor, a solution reactor, or a combination of two or more of these reactors. Representative slurry polymerization reactors and/or processes are disclosed, for example, in U.S. Pat. Nos. 3,248,179, 4,501,885, 5,565,175, 5,575,979, 6,239,235, 6,262,191, 6,833,415, and 8,822,608, and representative gas phase or fluidized bed reactors and/or processes are disclosed in U.S. Pat. Nos. 5,352,749, 4,588,790, 5,436,304, 7,531,606, and 7,598,327.

The polymerization conditions for the various reactor types are well known to those of skill in the art. Nonetheless, a suitable polymerization temperature can be any temperature below the de-polymerization temperature according to the Gibbs Free energy equation. Typically, this includes from 60° C. to 280° C., for example, or from 60° C. to 120° C., depending upon the type of polymerization reactor(s). In some reactor systems, the polymerization temperature generally can be within a range from 70° C. to 105° C., or from 75° C. to 100° C. Suitable pressures will also vary according to the reactor and polymerization type. The pressure for liquid phase polymerizations in a loop reactor is typically less than 1000 psig (6.9 MPa). Pressure for gas phase polymerization is usually from 200 to 500 psig (1.4 MPa to 3.4 MPa). High pressure polymerization in tubular or autoclave reactors is generally run at from 20,000 psig to 75,000 psig (138 MPa to 517 MPa). Polymerization reactors can also be operated in a supercritical region occurring at generally higher temperatures and pressures. Operation above the critical point of a pressure-temperature diagram (supercritical phase) can offer advantages to the polymerization reaction process.

Generally, the ethylene polymer produced in the processes can comprise an ethylene homopolymer and/or an ethylene/α-olefin copolymer in one aspect, and can comprise an ethylene homopolymer, an ethylene/1-butene copolymer, an ethylene/1-hexene copolymer, and/or an ethylene/1-octene copolymer in another aspect, and can comprise an ethylene/α-olefin copolymer and/or an ethylene terpolymer (e.g., ethylene with 1-butene and 1-hexene) in yet another aspect, and can comprise an ethylene/1-hexene copolymer in still another aspect.

Articles of manufacture can be formed from, and/or can comprise, the ethylene polymers of this invention and, accordingly, are encompassed herein. For example, articles which can comprise the polymers of this invention can include, but are not limited to, an agricultural film, an automobile part, a bottle, a container for chemicals, a drum, a fiber or fabric, a food packaging film or container, a food service article, a fuel tank, a geomembrane, a household container, a liner, a molded product, a medical device or material, an outdoor storage product (e.g., panels for walls of an outdoor shed), outdoor play equipment (e.g., kayaks, bases for basketball goals), a pipe, a sheet or tape, a toy, or a traffic barrier, and the like. Various processes can be employed to form these articles. Non-limiting examples of these processes include injection molding, blow molding, rotational molding, film extrusion, sheet extrusion, profile extrusion, thermoforming, and the like. Additionally, additives and modifiers often are added to these polymers in order to provide beneficial polymer processing or end-use product attributes. Such processes and materials are described in Modern Plastics Encyclopedia, Mid-November 1995 Issue, Vol. 72, No. 12; and Film Extrusion Manual—Process, Materials, Properties, TAPPI Press, 1992.

In some aspects of this invention, the article of manufacture can comprise any of ethylene polymers described herein, and the article of manufacture can be or can comprise a film, such as a blown film; alternatively, a pipe product; or alternatively, a blow molded product, such as a blow molded bottle.

Referring now to ethylene oligomerization, an oligomerization catalyst composition is contacted with the ethylene in an oligomerization reactor system under oligomerization conditions to produce the ethylene oligomers. Oligomerization catalyst compositions, oligomerization reactor systems and suitable reactor types, oligomerization conditions, and resulting ethylene oligomer products are well known to those of skill in the art. Briefly, an “oligomerization” process using ethylene as the monomer produces a mixture of products comprising at least 30 wt. %, 50 wt. %, 60 wt. %, or 70 wt. % oligomers having from 4 to 40 carbon atoms, or from 4 to 20 carbon atoms, such as a total amount of C₆ olefins and C₈ olefins of least 50 wt. %, 65 wt. %, 75 wt. %, or 80 wt. %.

Although not limited thereto, the oligomerization catalyst composition can be a chromium-based catalyst system. A particular example of an oligomerization catalyst composition can include a heteroatomic ligand chromium compound complex and an organoaluminum compound, or a heteroatomic ligand, a chromium compound, and an organoaluminum compound. Examples of representative and non-limiting oligomerization catalyst compositions—and ethylene oligomerization processes and reactor systems—include those disclosed in U.S. Patent Publication Nos. 2017/0081257, 2017/0341998, 2017/0341999, 2017/0342000, 2017/0342001, and 2016/0375431, and in U.S. Pat. Nos. 10,493,422, 10,464,862, 10,435,336, 10,689,312, and 10,807,921. Generally, the organoaluminum compound can be an aluminoxane, an alkylaluminum compound, or a combination thereof. Representative aluminoxanes include methylaluminoxane (MAO), ethylaluminoxane, modified methylaluminoxane (MMAO), n-propylaluminoxane, iso-propyl-aluminoxane, n-butylaluminoxane, sec-butylaluminoxane, iso-butylaluminoxane, t-butylaluminoxane, 1-pentylaluminoxane, 2-entylaluminoxane, 3-pentyl-aluminoxane, iso-pentyl-aluminoxane, neopentylaluminoxane, and the like, while representative alkylaluminums include trimethylaluminum, triethylaluminum, tripropylaluminum, tributylaluminum, trihexylaluminum, trioctylaluminum, and the like. Often, the Al to Cr molar ratio of the catalyst system can be in a range from 10:1 to 5,000:1, from 50:1 to 3,000:1, from 50:1 to 3,000:1, from 75:1 to 2,000:1, or from 100:1 to 1,000:1.

The oligomerization reactor in which the ethylene oligomer product is formed can comprise any suitable reactor, and non-limiting examples of reactor types can include a stirred tank reactor, a plug flow reactor, or any combination thereof; alternatively, a fixed bed reactor, a continuous stirred tank reactor, a loop reactor, a solution reactor, a tubular reactor, a recycle reactor, or any combination thereof. In an aspect, the oligomerization reactor system can have more than one reactor in series and/or in parallel and can include any combination of reactor types and arrangements. Moreover, the oligomerization process used to form the ethylene oligomer product can be a continuous process or a batch process, or any reactor or reactors within the oligomerization reaction system can be operated continuously or batchwise.

The oligomerization conditions for the various reactor types are well known to those of skill in the art. Nonetheless, a suitable oligomerization temperature typically falls within a range from 0 to 160° C., and more often, the oligomerization temperature is from 40 to 150° C., from 60 to 130° C., from 60 to 115° C., from 70 to 115° C., from 70 to 100° C., or from 75 to 95° C. Suitable pressures will also vary according to the reactor type, but generally, oligomerization pressures fall within a range from 50 psig to 3000 psig. More often, the pressure ranges from 200 psig to 2000 psig, from 400 psig to 1500 psig, from 600 psig to 2000 psig, from 600 psig to 1300 psig, from 700 psig to 1500 psig, or from 700 psig to 1200 psig.

The ethylene oligomer product can contain C₄+ hydrocarbons, and generally the vast majority of the ethylene oligomer product is C₆ olefins and/or C₈ olefins. Thus, the ethylene oligomers include C₆ olefins (e.g., 1-hexene), C₈ olefins (e.g., 1-octene), and C₁₀+ olefins. In an aspect, the major ethylene oligomer in the oligomer product is 1-hexene, while in another aspect, the major ethylene oligomer in the oligomer product is 1-octene, and in yet another aspect, the major ethylene oligomers in the oligomer product are 1-hexene and 1-octene (a mixture thereof).

As a general rule, the total amount of C₆ olefins and C₈ olefins—based on the total weight of oligomers in the ethylene oligomer product—can be at least 50 wt. %, and more often, at least 65 wt. %, at least 75 wt. %, or at least 85 wt. %, although not limited thereto. After the ethylene oligomer product is discharged in an effluent stream from the oligomerization reactor, the various components can be separated or fractionated into various ethylene oligomer product streams, such as a C₆ olefin product stream (e.g., containing predominantly 1-hexene), a C₈ olefin product stream (e.g., containing predominantly 1-octene), and so forth.

### Aspects

The invention is described above with reference to numerous aspects and specific examples. Many variations will suggest themselves to those skilled in the art in light of the above detailed description. All such obvious variations are within the full intended scope of the appended claims. Other aspects of the invention can include, but are not limited to, the following (aspects are described as “comprising” but, alternatively, can “consist essentially of” or “consist of”):

Aspect 1. A process (for converting ethane into ethylene) comprising (a) subjecting a water feed stream to electrolysis to form O₂ and H₂, (b) subjecting a mixture of ethane and (all or any portion of) the O₂ from step (a) to oxidative dehydrogenation to form a reaction product containing ethylene, acetic acid, water, and CO/CO₂, (c) separating the reaction product into an ethylene product stream, an acetic acid product stream, a water product stream, and a gas stream containing CO/CO₂, and (d) introducing (all or any portion of) the water product stream of step (c) into the water feed stream of step (a).

Aspect 2. The process defined in aspect 1, wherein the water product stream and a make-up water stream are combined in any suitable relative amount to form the water feed stream.

Aspect 3. The process defined in aspect 1 or 2, wherein the electrolysis utilizes a green source of electricity (e.g., wind energy, solar energy).

Aspect 4. The process defined in any one of the preceding aspects, wherein heat generated in the oxidative dehydrogenation is used in the electrolysis.

Aspect 5. The process defined in any one of the preceding aspects, wherein the process further comprises a step of contacting a polymerization catalyst composition with (all or any portion of) the ethylene product stream and an optional olefin comonomer in a polymerization reactor system under polymerization conditions to produce an ethylene polymer.

Aspect 6. The process defined in any one of the preceding aspects, wherein the process further comprises a step of contacting an oligomerization catalyst composition with (all or any portion of) the ethylene product stream in an oligomerization reactor system under oligomerization conditions to produce ethylene oligomers.

Aspect 7. The process defined in any one of the preceding aspects, wherein the process further comprises a step of reacting sulfur with (all or any portion of) the H₂ from step (a) to form H₂S.

Aspect 8. The process defined in any one of the preceding aspects, wherein the process further comprises a step of reacting sulfur with (a portion of) the O₂ from step (a) to form SO₂ and/or a step of reacting methyl mercaptan with (a portion of) the O₂ from step (a) to form dimethyl disulfide.

Aspect 9. The process defined in any one of the preceding aspects, wherein the process further comprises a step of reacting (all or any portion of) the gas stream containing CO/CO₂ from step (c) with (all or any portion of) the H₂ from step (a) to form methanol.

Aspect 10. The process defined in any one of the preceding aspects, wherein step (c) comprises separating the reaction product into the ethylene product stream, a mixture of acetic acid and water, and the gas stream containing CO/CO₂, and separating the mixture into the acetic acid product stream, the water product stream, and steam.

Aspect 11. The process defined in aspect 10, wherein (all or any portion of) the steam is utilized in the oxidative dehydrogenation of step (b).

Aspect 12. The process defined in any one of the preceding aspects, wherein the process further comprises a step of processing air through an air separation unit to form N₂ and O₂.

Aspect 13. The process defined in aspect 12, wherein (all or any portion of) the O₂ is used in the oxidative dehydrogenation of step (b).

Aspect 14. The process defined in aspect 12 or 13, wherein the process further comprises a step of contacting (all or any portion of) the H₂ from the electrolysis of step (a), (all or any portion of) the N₂ from the air separation unit, and an ammonia synthesis catalyst to form ammonia.

Aspect 15. The process defined in aspect 14, wherein the process further comprises a step of contacting (all or any portion of) CO₂ from the gas stream containing CO/CO₂ and (all or any portion of) the ammonia to form urea.

Aspect 16. The process defined in any one of the preceding aspects, wherein the process further comprises a step of contacting (all or any portion of) CO₂ from the gas stream containing CO/CO₂ of step (c), (all or any portion of) the H₂ from the electrolysis of step (a), (all or any portion of) the acetic acid product stream of step (c), and a catalyst to from ethanol.

Aspect 17. The process defined in aspect 16, wherein the process further comprises a step of contacting (all or any portion of) the ethanol with a catalyst to produce a reaction mixture containing ethylene and water.

Aspect 18. The process defined in aspect 17, wherein the process further comprises a step of separating the reaction mixture into an ethylene stream and a water stream.

Aspect 19. The process defined in aspect 18, wherein the process further comprises a step of combining (all or any portion of) the ethylene stream with the ethylene product stream of step (c).

Aspect 20. The process defined in aspect 18 or 19, wherein the process further comprises a step of combining (all or any portion of) the water stream with the water feed stream of step (a).

Aspect 21. The process defined in any one of the preceding aspects, wherein the process further comprises a step of subjecting (all or any portion of) the CO/CO₂ stream to carbon capture and storage (CCS) to isolate CO₂.

Aspect 22. The process defined in any one of the preceding aspects, wherein the process further comprises a step of gasifying a mixture of any portion of the O₂ from step (a) and a plastic (biomass, mixed solid waste, or a combination thereof) to form a Syngas stream.

Aspect 23. The process defined in any one of the preceding aspects, wherein the process further comprises a step of gasifying a mixture of any portion of the O₂ from step (a) and a plastic (biomass, mixed solid waste, or a combination thereof) and Fischer-Tropsch process to produce alkanes (e.g., for fuel applications).

Aspect 24. The process defined in any one of the preceding aspects, wherein the process further comprises a step of gasifying a mixture of any portion of the O₂ from step (a) and a plastic (biomass, mixed solid waste, or a combination thereof) to form a Syngas stream, and reacting (all or any portion of) the gas stream containing CO/CO₂ from step (c) with all or a portion of the H₂ from step (a) to form methanol.

Aspect 25. The process defined in any one of the preceding aspects, wherein the process further comprises the steps of (i) gasifying a mixture of any portion of the O₂ from step (a) and a plastic to form a Syngas stream, (ii) separating CO and H₂ from the Syngas stream, and (iii) contacting the CO, H₂, and a multicomponent catalyst to form a reaction mixture containing ethanol.

Aspect 26. The process defined in any one of aspects 5-25, wherein the polymerization catalyst composition is a metallocene catalyst system, a Ziegler-Natta catalyst system, a chromium catalyst system, or any combination thereof.

Aspect 27. The process defined in any one of aspects 5-26, wherein the ethylene polymer comprises an ethylene homopolymer and/or an ethylene/α-olefin copolymer.

Aspect 28. The process defined in any one of aspects 5-27, wherein the ethylene polymer comprises an ethylene homopolymer, an ethylene/1-butene copolymer, an ethylene/1-hexene copolymer, and/or an ethylene/1-octene copolymer.

Aspect 29. The process defined in any one of aspects 6-25, wherein the oligomerization catalyst composition comprises a heteroatomic ligand transition metal compound complex and an organoaluminum compound, or a heteroatomic ligand, a transition metal compound, and an organoaluminum compound.

Aspect 30. The process defined in any one of aspects 6-29, wherein the ethylene oligomers comprise C₆ olefins (e.g., 1-hexene), C₈ olefins (e.g., 1-octene), and C₁₀+ olefins.

Aspect 31. A process (for converting methane) comprising (A) subjecting a water feed stream to electrolysis to form O₂ and H₂, (B) subjecting a mixture of methane and (all or any portion of) the O₂ from step (A) and CO₂ to autothermal reforming to form a reaction product containing H₂, CO, and water, (C) separating the reaction product into a H₂ product stream, a water product stream, and a gas stream containing CO/CO₂, and (D) introducing (all or any portion of) the water product stream of step (C) into the water feed stream of step (A).

Aspect 32. A process comprising a) subjecting a water feed stream to electrolysis to form O₂ and H₂, and b) gasifying a mixture of a plastic and (all or any portion of) the O₂ from step a) to form a Syngas stream.

We claim:

1. A process comprising:
(a) subjecting a water feed stream to electrolysis to form O₂ and H₂;
(b) subjecting a mixture of ethane and all or any portion of the O₂ from step (a) to oxidative dehydrogenation to form a reaction product containing ethylene, acetic acid, water, and CO/CO₂;
(c) separating the reaction product into an ethylene product stream, an acetic acid product stream, a water product stream, and a gas stream containing CO/CO₂; and
(d) introducing all or any portion of the water product stream of step (c) into the water feed stream of step (a).

2. The process of claim 1, wherein the water product stream and a make-up water stream are combined to form the water feed stream.

3. The process of claim 1, wherein the electrolysis utilizes a green source of electricity.

4. The process of claim 1, wherein heat generated in the oxidative dehydrogenation is used in the electrolysis.

5. The process of claim 1, wherein the process further comprises a step of contacting a polymerization catalyst composition with all or any portion of the ethylene product stream and an optional olefin comonomer in a polymerization reactor system under polymerization conditions to produce an ethylene polymer.

6. The process of claim 5, wherein:
the polymerization catalyst composition is a metallocene catalyst system, a Ziegler-Natta catalyst system, a chromium catalyst system, or any combination thereof; and
the ethylene polymer comprises an ethylene homopolymer, an ethylene/1-butene copolymer, an ethylene/1-hexene copolymer, and/or an ethylene/1-octene copolymer.

7. The process of claim 1, wherein the process further comprises a step of contacting an oligomerization catalyst composition with all or any portion of the ethylene product stream in an oligomerization reactor system under oligomerization conditions to produce ethylene oligomers.

8. The process of claim 7, wherein:
the oligomerization catalyst composition comprises a heteroatomic ligand transition metal compound complex and an organoaluminum compound, or a heteroatomic ligand, a transition metal compound, and an organoaluminum compound; and
the ethylene oligomers comprise C₆ olefins, C₈ olefins, and C₁₀+ olefins.

9. The process of claim 1, wherein the process further comprises a step of reacting sulfur with all or any portion of the H₂ from step (a) to form H₂S.

10. The process of claim 1, wherein the process further comprises:
a step of reacting sulfur with a portion of the O₂ from step (a) to form SO₂; and/or
a step of reacting methyl mercaptan with a portion of the O₂ from step (a) to form dimethyl disulfide.

11. The process of claim 1, wherein the process further comprises a step of reacting all or any portion of the gas stream containing CO/CO₂ from step (c) with all or any portion of the H₂ from step (a) to form methanol.

12. The process of claim 1, wherein step (c) comprises:
separating the reaction product into the ethylene product stream, a mixture of acetic acid and water, and the gas stream containing CO/CO₂; and
separating the mixture into the acetic acid product stream, the water product stream, and steam.

13. The process of claim 12, wherein all or any portion of the steam is utilized in the oxidative dehydrogenation of step (b).

14. The process of claim 1, wherein the process further comprises a step of processing air through an air separation unit to form N₂ and O₂.

15. The process of claim 14, wherein:
all or any portion of the O₂ is used in the oxidative dehydrogenation of step (b);
the process further comprises a step of contacting all or any portion of the H₂ from the electrolysis of step (a), all or any portion of the N₂ from the air separation unit, and an ammonia synthesis catalyst to form ammonia; and
the process further comprises a step of contacting all or any portion of CO₂ from the gas stream containing CO/CO₂ and all or any portion of the ammonia to form urea.

16. The process of claim 1, wherein the process further comprises a step of contacting all or any portion of CO₂ from the gas stream containing CO/CO₂ of step (c), all or any portion of the H₂ from the electrolysis of step (a), all or any portion of the acetic acid product stream of step (c), and a catalyst to from ethanol.

17. The process of claim 16, wherein the process further comprises:
a step of contacting all or any portion of the ethanol with a catalyst to produce a reaction mixture containing ethylene and water;
a step of separating the reaction mixture into an ethylene stream and a water stream;
a step of combining all or any portion of the ethylene stream with the ethylene product stream of step (c); and
a step of combining all or any portion of the water stream with the water feed stream of step (a).

18. The process of claim 1, wherein the process further comprises:
a step of subjecting all or any portion of the CO/CO₂ stream to carbon capture and storage (CCS) to isolate CO₂;
a step of gasifying a mixture of any portion of the O₂ from step (a) and a plastic to form a Syngas stream;
a step of gasifying a mixture of any portion of the O₂ from step (a) and a plastic and Fischer-Tropsch process to produce alkanes;
a step of gasifying a mixture of any portion of the O₂ from step (a) and a plastic to form a Syngas stream, and reacting all or any portion of the gas stream containing CO/CO₂ from step (c) with all or any portion of the H₂ from step (a) to form methanol; or
any combination thereof.

19. The process of claim 1, wherein the process further comprises the steps of:
(i) gasifying a mixture of any portion of the O₂ from step (a) and a plastic to form a Syngas stream;
(ii) separating CO and H₂ from the Syngas stream; and
(iii) contacting the CO, H₂, and a multicomponent catalyst to form a reaction mixture containing ethanol.

20. A process comprising:
(A) subjecting a water feed stream to electrolysis to form O₂ and H₂;
(B) subjecting a mixture of methane and all or any portion of the O₂ from step (a) and CO₂ to autothermal reforming to form a reaction product containing H₂, CO, and water;
(C) separating the reaction product into a H₂ product stream, a water product stream, and a gas stream containing CO/CO₂; and
(D) introducing all or any portion of the water product stream of step (C) into the water feed stream of step (A).

21. A process comprising:
a) subjecting a water feed stream to electrolysis to form O₂ and H₂; and
b) gasifying a mixture of a plastic and all or any portion of the O₂ from step a) to form a Syngas stream.
